(19) United States
(12) Patent Application Publication
(10) Pub. No.: US 2005/0053663 A1
(43) Pub. Date: Mar. 10, 2005

(54) Chitosan-calcium complex and methods of producing the complex

(76) Inventors: Henryk Struszczyk, Zgierz (PL); Antoni Niekraszewicz, Lodz (PL); Magdelena Kucharska, Lodz (PL); Alojzy Urbanowski, Lodz (PL); Kinga Brzoza, Swidnica (PL); Danuta Ciechanska, Lodz (PL); Maria Wisniewska-Wrona, Lodz (PL)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/501,173

(22) PCT Filed: Jan. 8, 2003
(86) PCT No.: PCT/IB03/00024

(30) Foreign Application Priority Data
Jan. 9, 2002 (PL) P351602
Jan. 9, 2002 (PL) P351603

(51) Int. Cl. C08B 37/08; A61K 9/14

(52) U.S. Cl. 424/488; 514/55; 536/20

(57) ABSTRACT

Chitosan-calcium complexes of calcium (II) ions with a gel of a chitosan salt, containing not less than 0.5 wt % of the polymer with an average molecular weight not less than 10 kD, a polydispersity degree not lower than 2.0 and a deacetylation degree at least 65%. The complex is characterized by pH not higher than 6.9 and a calcium Ca (II) ions content not less than 0.1 wt % on chitosan. Methods to prepare chitosan-calcium complexes use a gel of a chitosan salt, containing not less than 0.5 wt % of the polymer. Calcium salts are introduced in the amount of at least 0.1 wt % Ca (II) on chitosan weight. The mixture is next homogenized and reacted at 10° C. during a time not shorter than 1 minute. Methods of producing a gel of chitosan salts involves subjecting a chitosan with the concentration of at least 0.5 wt % in an aqueous acidic solution to a controlled enzymatic, hydrolytic or oxidative degradation.

### FIELD OF THE INVENTION

The invention concerns a chitosan-calcium complex and a method to produce the complex. The invention also concerns a method to produce modified gel of chitosan salts.

### BACKGROUND OF THE INVENTION

The sorption phenomenon of metal ions by chitosan in its solid state or solutions in aqueous organic and inorganic acid is well-known, as exemplified by International Journal of Biological Macromolecules, v.9, p. 109, 1987, “Carbohydrate Polymer”, v. 8, p. 1-21, 1988, v.11, p. 205-307, 1989; “Talanta”, v. 16, p. 1571-1579, 1969; “Carbohydrate Polymers”, v. 36, p. 267-276, 1998 and monographs “Chitin Chemistry”, MacMillan Press Ltd, Great Britain, 1992, p. 222-225 and “Advances in Chitin Science”, V. IV, Universitat Potsdam, Germany, 2000, p. 202-205.

The amount of bound calcium Ca (II) ions is insignificant compared to other alkali metals, amounting to only 0.4-0.8×10⁻³ mol/gr of chitosan. Soluble derivatives of chitosan demonstrate a better ability to bind calcium Ca (II) ions notably carboxymethylchitosan, carboxybenzylchitosan, (N)methylchitosan phosphoniate. Complexes of these derivatives with calcium Ca (II) ions do not dissolve in water. Unknown are chitosan complexes with calcium Ca (II) ions able to dissolve in water or to produce thermally stable suspensions. The complex, according to the invention, is a compound of calcium (II) ions with the gel of chitosan salts, containing at least 0.5 wt % of the polymer characterized by: average molecular weight—not lower than 10 kD, polydispersity degree—not lower than 2.0 and deacetylation degree—at least 65%. The complex is characterized by pH—not exceeding 6.9, content of Calcium (II) ions not lower than 0.1 wt % on chitosan.

Chemical methods to produce a gel of chitosan salts by reacting organic- and/or inorganic acids chitosan salts, with dialdehydes like glutaric aldehyde or with epioxides like epichlorhydrin are well-known, as exemplified by: “Die Makromolekulare Chemie”, v.190, p. 951-960, 1989; “Polimo”, v.14 (5), p. 516-526, 1990, “Advances in Chitin Science”, v.11, J. Andre Publisher, Lyon, France, 1998, p. 484-491; “Advances in Chitin Science”, v.IV, Universitat Potsdam, Germany, 2000, p. 98-103; “Chitin Chemistry”, Mac Millan Press Ltd., Great Britain, 1992, p. 305-315 and U.S. Pat. No. 6,277,792 and U.S. Pat. No. 6,314,045.

Chemical methods to produce chitosan gel by acylation of chitosan with anhydrides of organic acids like acetic acid anhydride in a medium containing water, acetic acid and alcohols are well-known, as exemplified by: “Angewande Makromolekulare Chemie”, v. 207, p.1, 1993; “International Journal of Biological Macromolecules”, v.2, p. 73-77, 1980; “Die Makromolekular Chemie”, v.190, 1989; “Polymer”, v. 16, p.622, 1975; “Carbohydrate Research”, v. 47, p. 315, 1976 and monograph “Chitin”, Pergamon Press, Oxford, 1997, p.134 and “Advances in Chitin Science”, v. II, J. Andre Publisher, Lyon, France, 1998, p. 453-461 and 339-348.

Physico-chemical methods to produce chitosan gel by dissolving the chitosan in an aqueous solution of a dicarboxylic acid like oxalic acid or preparing a gel in the medium of polyoxy anions of molybdenum salts are known from following publications: “Carbohydrate Research”, v.201, p.145-149, 1990; “Biomaterials”, v.13 (9), p. 635-638, 1976, v.15, p. 1685-1691, 1976 and the monograph “Advances in Chitin Science”, v. IV, Universitat Potsdam, Germany, 2000, p. 98.

Methods to produce chitosan gel in the presence of multihydroxide alcohols like glycol, glycerol as a complex with poly(vinyl-lactames), alginates, carboxmethylcellulose, polymethacrylates, proteins or xanthan are well known. As described in U.S. Pat. Nos. 4,659,700, 5,037,664, 5,098,733, 5,395,305, 5,382,286, 5,420,197, 5,620,706 and 5,836,970.

A method to produce chitosan gel by treating chitosan lactate with papain to obtain a low-molecular weight product is well-known from the monograph “Chitin Enzymology”, Atec Edizioni Publ., Ancona, Italy, 2001, p.409 and from the publication in “Carbohydrate Polymers”, V. 29, p. 63-68, 1999.

Gel agents, improving the immunity of plants against pathogens and biostimulating the plants, produced by a partial neutralization of a chitosan solution in organic acids with sodium hydroxide or potassium hydroxide or sodium carbonate to a pH of 5.0-6.6 are known from International Patent Applications WO 970987 and WO 8901288 and abstract of the Japanese Patent JP 03133909. Polish Patent Application P340131 and International Application WO 01/87067 claim a chitosan gel, applied for biostimulating the growth of plants, formed in a step-wise production of microcrystalline chitosan as a result of partial neutralization of an aqueous solution of chitosan salts with the concentration of at least 0.001% by means of hydroxides with a concentration within 0.01-2% to attain pH=5.0-6.9 with agitation during at least 10 seconds with a shearing force of 10-1000 sec⁻¹.

The well-known methods to produce chitosan gel do not achieve the manufacture of a product with controlled structure mainly molecular one and assumed properties. In these methods it is necessary to use additional substances, capable of modifying cross-linkages or cause a secondary acetylation, thus producing a chitin gel. The known methods do not enable the forming of a durable chitosan salt gel, which could be diluted with water or aqueous solutions of organic/inorganic acids. These methods also do not produce a thermally stable gel.

### SUMMARY OF THE INVENTION

These and other issues are addressed by the present invention.

The present invention relates to a chitosan-calcium (II) complex having calcium (II) ions bound to a gel of a chitosan salt, wherein the complex contains ≧0.5 wt % chitosan having an average molecular weight≧10 kD, a polydispersity≧2.0, deacetylation degree≧65% and where the complex has a water retention value≧300%, pH≦6.9 and a calcium (II) ion content≧0.1 wt % relative to chitosan.

According to another aspect, the invention relates to methods of producing a chitosan-calcium complex, having a water retention value≧300% and a pH≦6.9, from a gel of a chitosan salt. The suspension contains ≧0.01 wt % chitosan gel having an average polymerization degree≧10 kD, a polydispersity≧2.0, and deacetylation degree≧65%. The gel is mixed with ≧0.01 wt % calcium (II) salt to form the complex.

The present invention also relates to methods for preparing chitosan salt gels in which chitosan is enzymatically degraded in an aqueous acidic solution. The solution has a chitosan concentration≧0.5 wt %. Then the enzymes are deactivated at a desired time and an aqueous basic solution is added to the enzyme/aqueous chitosan mixture to attain 4.0≦pH≦6.0. The mixture is continuously mixed until a gel of a chitosan salt forms.

In another aspect, methods of the invention can be carried out by preparing a gel of a chitosan salt by hydrolytic degradation of chitosan in aqueous acidic solution. The solution has a chitosan concentration≧0.5 wt %. Aqueous base is added to the mixture to attain 4.0≦pH≦6.0 and then the product is mixed continuously until a gel of a chitosan salt forms.

In another aspect, methods of the invention can be carried out using an oxidizing agent to degrade the chitosan.

### DESCRIPTION OF THE INVENTION

According to the invention, calcium Ca (II) ions are advantageously linked by coordinate and/or secondary bonds like hydrogen bonds in a complex with chitosan.

According to one aspect, methods according to the invention include introducing calcium Ca (II) salts like calcium chloride or calcium acetate in the amount of at least 0.1 wt %, preferably 10-50 wt % on chitosan weight to a gel of a chitosan salt, containing at least 0.5 wt of the polymer with an average polymerization degree not lower than 10 kD, a deacetylation degree of at least 65%, a polydispersity degree not lower than 2.0 and a pH not exceeding 6.9. The mixture is then homogenized and reacted for not less than 1 minute, preferably 30-120 minutes, at a temperature not lower than 10° C.

The obtained complex may be concentrated and dried according to known methods.

Formation of the complex may be carried out in two steps. The initial step of mixing the chitosan salt gel with calcium salts runs at an agitation speed below 100 rpm and the consecutive step of forming the complex at 100-10000 rpm.

The chitosan/calcium complex, according to the invention, is characterized by the presence of mainly coordinate bonds between the calcium Ca (II) ions and the amide and hydroxide groups of the chitosan and intra- and intermolecular hydrogen bonds between the amide, amino and hydroxide groups of the chitosan molecule chain.

These bonds are characterized by a high energy exceeding the known forms of chitosan. The bonds contribute to stabilizing the chitosan-calcium complex structure, resulting in a high stability and high content of calcium ions.

A further advantage of the invention is the simple way of producing the chitosan-calcium complex with unique properties: high content of bound calcium Ca (II) ions, stability also at high temperature and biological activity. An advantage of the chitosan-calcium complex is its bioactivity, superior in comparison with other known forms of chitosan.

The chitosan-calcium complex of the invention is applied mainly in medicine and pharmacy. The invention is illustrated with following examples, which do not limit its range of application.

### EXAMPLE 1

To a mixer equipped with a slow/fast agitation system, 120 wt parts of a chitosan salt gel with modified structure, characterized by: polymer content −0.72 wt %, Mᵥ=408 kD, P_{d}=2.43, DD=85.6%, pH=6.48 were introduced followed by step-wise feeding, during 10 minutes, of 2.0 wt parts of calcium chloride at constant agitation with 80 rpm.

Mixing proceeded at 25° C. for 5 minutes at 40 rpm and, next, for 10 minutes, the chitosan-calcium complex was formed at 8000 rpm. The product was steam-sterilized for 30 minutes at 121° C.

122 wt parts of chitosan-calcium complex as a stable gel were obtained, containing 1.69 wt % of the polymer, characterized by Mᵥ=405 kD, P_{d}=2.33, DD=85.6% and 21.3% content of calcium (II), on chitosan weight.

### EXAMPLE 2

To the mixer, as in Example 1, 120 wt parts of a chitosan salt gel, characterized by: polymer content −1.65 wt %, Mᵥ=600 kD, P_{d}=3.59, DD=82.2% and pH=6.45 were introduced followed by a step-wise feeding, during 10 minutes, with 0.5 wt part of calcium chloride at constant agitation at 80 rpm. Mixing proceeded at 28° C. for 5 minutes at 40 rpm, then, during 10 minutes the chitosan-calcium complex was formed at 8000 rpm. The product was steam-sterilized during 30 minutes at 121° C.

120.5 wt parts were obtained of chitosan-calcium complex as a stable gel, containing 1.64 wt % of polymer with Mᵥ=582 kD, P_{d}=3.50, DD=82.2%, pH=6.40 and 6.1 wt % content of calcium Ca (II), on chitosan weight.

### EXAMPLE 3

To the mixer, as in Example 1, 150 wt parts of the chitosan salt gel with modified molecular structure were introduced. The chitosan gel was characterized by: polymer content of 1.85 wt %, Mᵥ=320 kD, P_{d}=2.72, DD=80.2% and pH=6.60. Next, the mixer was step-wise fed, during 10 minutes, with 2.5 wt parts of calcium chloride at 40 rpm of the agitator at 30° C. During a next ten minutes the chitosan-calcium complex was formed at 8000 rpm of the agitator. The obtained product was steam-sterilized during 30 minutes at 121° C.

152.5 wt parts were obtained of a chitosan-calcium complex as a stable gel containing 1.82 wt % of polymer with Mᵥ=305 kD, P_{d}=2.78, DD=80.2%, pH=6.45 and 19.8% content of calcium (II), on weight of chitosan.

### EXAMPLE 4

To the mixer as in Example 1, 100 wt parts of a modified chitosan gel were introduced. The chitosan gel was characterized by polymer content of 2.8 wt %, Mᵥ=150 kD, P_{d}=3.42, DD=85.6% and pH=6.71. Next, the mixer was fed with 10 wt parts of a 10% aqueous solution of calcium acetate while mixing at 80 rpm. The mixer content was next agitated for 10 minutes at 15° C. During a next 5 minutes the chitosan-calcium complex was formed at agitation speed of 8500 rpm. 110 wt parts were obtained of a chitosan-calcium complex in the form of a stable gel containing 2.55 wt % of chitosan with Mᵥ=145 kD, P_{d}=3.40, DD=85.6% and pH=6.60, and 5.80 wt % content of calcium (II), on weight of chitosan.

The method to produce a modified chitosan gel, according to the invention, involves treating chitosan in aqueous solution, with the polymer concentration not less than 0.5%, preferably 1-3%, to a controlled enzymatic degradation. The degradation is accomplished with the use of cellulases, chitanases or xylanases during a time of 1 minute to 100 hours at a temperature not lower than 10° C., preferably 20-60° C., with the enzyme activity not less than 0.01 units/cm³. After the enzymatic treatment, the remaining enzymes are deactivated at a temperature above 70° C. The chitosan solution is then partially neutralized with aqueous hydroxides or their salts with a 5-10% concentration at vigorous agitation with no more than 5000 rpm to attain pH=4.0-6.0. Afterwards, the reaction mixture is continuously agitated at a speed below 10 000 rpm to reach the gel-forming point, corresponding to pH=6.3-6.9. The obtained modified gel of the chitosan salt may be dried in a classical way.

An another version of the invention consists in the hydrolytic degradation of the chitosan in its aqueous solution with a not less than 0.5%, preferably 1-3% polymer concentration. The hydrolytic degradation proceeds for between 1 minute to 100 hours at not lower than 20° C., preferably 40-80° C., preferably in the presence of strong acids like hydrochloric or chloroacetic acids used in the amount of at least 0.01% on the polymer. After the degradation, the chitosan solution is partially neutralized with aqueous hydroxides or their salts with a concentration of 5-10% at vigorous agitation with a speed not exceeding 5000 rpm to attain pH=4.0-6.0. After that, the reaction mixture is still agitated with a speed not exceeding 10000 rpm till the gel-forming point is reached corresponding to pH=6.3-6.9. The resulting modified gel of the chitosan salt is possibly dried in a classical way.

Another version of the invention consists in the oxidative degradation of the chitosan in its aqueous solution with not less than 0.5%, preferably 1-3%, concentration of the polymer. The oxidative degradation is accomplished by the use of an oxidizing agent like hydrogen peroxide or sodium perborate in the amount of not less than 0.01-0.5% on chitosan. The chitosan solution is then partially neutralized with aqueous hydroxides or their salts with a concentration of 5-10% at vigorous agitation with no more than 5000 rpm to attain pH=4.0-6.0. The reaction mixture is still agitated with a speed not exceeding 10000 rpm until the gel-forming point is reached corresponding to pH=6.3-6.9. The resultant, modified gel of chitosan may be dried in a classical way.

According to the invention, aqueous solutions of chitosan in hydrochloric, acetic or lactic acid may be used.

For neutralizing, aqueous solutions of sodium or potassium hydroxides are applied or their salts like sodium or potassium carbonate.

A batch-wise or continuous process can be utilized for the manufacture of the modified gel of chitosan salt.

In one aspect of the invention with enzymatic degradation allows control of two processes—statistical degradation and depolymerization—thus enabling manufacture of a chitosan salt gel with assumed content of bioactive oligoaminosaccharides and reduced polydispersity.

Products resulting from the embodiments of the invention employing hydrolytic or oxidative degradation were subjected to mainly statistical degradation. One of the advantages of the process according to the invention is the formation of macromolecules with assumed molecular characteristics, enhancing the manufacture of a gel-like product. For example, chemical modification or sophisticated combination of dicarboxylic acids and mixed organic/inorganic solvent is not necessary. The gel of the chitosan salt is formed in a homogeneous phase as a result of the presence of strong ionic and secondary bonds like hydrogen bonds which contribute to a stable gel form in the pH range of 6.3-6.9. More particularly, an advantage of the method according to the invention is the ability to produce a gel of chitosan salts with a controlled primary structure, like average molecular weight, polydispersity degree and assumed physico-chemical properties like stability of the gel form, viscosity, gel rheology and assumed useful properties like bioactivity, biodegradability and ability to sustain diluting with water or aqueous acids. The chitosan salt gel is thermally stable thus enabling, amongst other, thermal sterilization above 100° C. for medical applications and, besides, it is not prone to dehydration during at least 1-2 years.

Gels of chitosan salts produced according to the invention are useful in medicine and pharmacy.

The method according to the invention is illustrated with the following examples, which do not limit its range of application.

### EXAMPLE 5

5175 wt parts of a 0.5% aqueous hydrochloric acid and 100 wt parts of the initial chitosan powder were introduced to a reactor equipped with an agitator and cooling jacket. The chitosan was characterized by: average molecular weight Mᵥ=740 kD, polydispersity degree P_{d}=3.68, deacetylation degree DD=85.7%, content of insoluble parts −0.3% and moisture 10.2%. The dissolution proceeded for 2 hours with the agitator at 50 rpm. The product chitosan solution was filtered using a frame filter press. 5260 wt parts of a solution containing 1.7 wt % of chitosan were obtained.

The solution was introduced to a mixer equipped with slow/fast agitator system. 180 wt parts of a 10% aqueous sodium hydroxide were introduced at 20° C. to the mixer at 4000 rpm of the agitator to attain pH=5.25. Next, at 30 rpm of the agitator 0.26 wt part was introduced of the enzyme—Ekonaza CE with the initial endo-1,4-β-glucanase activity equal to 2600 U CMC/cm³. The enzyme activity in the reaction solution was 0.13 U CMC/cm³. The controlled enzymatic degradation was conducted at 20° C. for 40 minutes, then the temperature of the reaction mixture was raised to 80° C. for 10 minutes to deactivate the remaining enzymes. The reaction mixture was then cooled to 21° C. for 15 minutes. The 10% aqueous sodium hydroxide were introduced to attain pH=6.0. The agitation was continued at 8000 rpm to reach the gel-forming point at pH=6.52.

5510 wt parts of a stable gel of the chitosan salt were obtained with a jelly consistency containing 1.62 wt % of chitosan, characterized by Mᵥ=550 kD, P_{d}=3.06, DD=85.7%.

### EXAMPLE 6

4459 wt parts of a 2.0% aqueous acetic acid and 100 wt parts of the initial chitosan flakes were introduced to the reactor as in Example 5. The chitosan was characterized by: Mᵥ=138 kD, P_{d}=2.98, DD=75.0%, insoluble part content was 0.1% and moisture was 8.9%. The dissolution proceeded for 2 hours with agitator speed of 50 rpm. The chitosan solution was filtered using a frame filter press.

4545 wt parts containing 2.0 wt % of chitosan were obtained. The solution was introduced to a mixer equipped with a slow/fast agitator system. At 4000 rpm and 20° C., 98 wt parts of a 10% aqueous sodium hydroxide were introduced to the mixer to attain pH=5.25. Next, at the agitator speed of 30 rpm 0.22 wt part of the enzyme Ekonaza CE was introduced. The initial endo-1,4-β-glucanase activity of the enzyme was equal to 2600 U CMC/cm³ while in the reaction mixture the activity was 0.13 U CMC/cm³. The controlled enzymatic degradation was run for 20 minutes at 20° C., then the temperature was raised to 80° C. for 10 minutes to deactivate the remaining enzyme. Afterwards, the mixer content was intensively cooled to 21° C. during 15 minutes and agitated with the agitator speed 5000 rpm. At this speed and temperature of 20±1° C., 42 parts of a 10% aqueous sodium hydroxide were then introduced to attain pH=6.0, then the agitator speed was raised to 8000 rpm and kept to attain the gel-forming point at pH=6.69.

4680 wt parts of a stable gel of chitosan salt with a jelly consistency were obtained; the gel contained 1.94 wt % chitosan, characterized by Mᵥ=74 kD, P_{d}=3.05, DD=75.0%.

### EXAMPLE 7

4459 wt parts of 1% aqueous lactic acid and 100 wt parts of chitosan flakes were introduced to the reactor as in Example 1. The properties of the chitosan were the same as in Example 6. The dissolution proceeded for 2 hours with agitator speed of 50 rpm. The chitosan solution was filtered using a frame filter press obtaining 4545 wt parts of a solution containing 2% chitosan. The solution was introduced to a mixer equipped with a slow/fast agitator system. To the mixer with agitator speed 4000 rpm, 83 wt parts of a 10% aqueous potassium hydroxide were introduced at 20° C. to attain pH=5.25. Next, at the agitator speed of 30 rpm 0.23 wt part of the enzyme—Ekonaza was introduced. The enzyme endo-1,4-β-glucanase initial activity was 2600 U CMC/cm³ while in the reaction mixture it was 0.13 U CMC/cm³. The controlled enzymatic degradation was run for 15 minutes at 20° C. Temperature was then raised to 80° C. for 10 minutes to deactivate the remaining enzyme. Then the mixer content was intensively cooled for 15 minutes to 20° C. Next, with the agitator at 5000 rpm and temperature still 20° C., 81 wt parts of a 10% aqueous sodium hydroxide were introduced to attain pH=6.0. Then the agitator speed was increased to 8000 rpm and maintained to reach the gel-forming point at pH=6.71.

4709 wt parts of a stable gel of chitosan salt with a jelly consistency was obtained; the gel contained 1.93% of chitosan, characterized by Mᵥ=78 kD, P_{d}=3.05, DD=75.0%.

### EXAMPLE 8

4439 wt parts of 0.5% aqueous hydrochloric acid and 100 wt parts of powdered initial chitosan were introduced to the reactor as in Example 5. The chitosan was characterized by: Mᵥ=346 kD, P_{d}=3.45, DD=82.2%, content of insoluble parts 0%, moisture 9.4%. The dissolution proceeded for 2 hours with agitation speed of 50 rpm. The product solution was filtered using a frame filter press obtaining 4530 wt parts of a solution containing 2% chitosan. The solution was introduced to a mixer equipped with a slow/fast agitator system and was, for 3 hours at 50±1° C. with the agitation speed of 400 rpm, subjected to a controlled hydrolytic degradation. The chitosan solution was next cooled to 20±1° C. At 4500 rpm, 115 wt parts of a 10% aqueous potassium hydroxide were introduced to attain pH=6.0. Afterwards, agitation speed was increased to 8000 rpm till the gel-forming point at pH=6.52 was reached.

4642 wt parts of a stable gel of the chitosan salt with a jelly consistency were obtained; the gel contained 1.95 wt % of chitosan, characterized by Mᵥ=295 kD, P_{d}=3.62, DD=82.2%.

### EXAMPLE 9

4459 wt parts of a 1.0% aqueous lactic acid and 100 wt parts of the initial chitosan flakes were introduced to the reactor as in Example 5. The chitosan was characterized as in Example 2. The dissolution was run for 2 hours at 50 rpm of the agitator, the product solution was filtered, using a frame filter press. 4545 wt parts of a solution, with the content of 2 wt % of chitosan were obtained. The solution was introduced to a mixer equipped with a slow/fast agitator system. The solution was next subjected to a hydrolytic degradation over 2 hours at 60±1° C. with the agitator speed of 200 rpm. Afterwards, the mixture was cooled to 20±1° C. and, at 4500 rpm of the agitator, 93 wt parts of a 10% aqueous potassium hydroxide introduced to attain pH=6.0. The agitator speed was then raised to 8000 rpm till the gel-forming point was reached at pH=6.69.

4637 parts of a stable gel of the chitosan salt with a jelly consistency was obtained; the gel contained 1.96 wt % of chitosan, characterized by: Mᵥ=92 kD, P_{d}=3.07, DD=75.0%.

### EXAMPLE 10

4351 wt parts of a 2.0% aqueous acetic acid and 100 wt parts of the initial chitosan in flakes were introduced to the reactor as in Example 5. The chitosan was characterized by: Mᵥ=520 kD, P_{d}=3.89, DD=78.0%, a content of insoluble parts of 0.4% and a moisture of 10.8%. Dissolution proceeded for 2 hours at the agitator speed of 50 rpm. Product solution was filtered using a frame filter press and obtaining 4440 wt parts of a solution, containing 2% chitosan. The solution was introduced to a mixer equipped with a slow/fast agitator system and, over 5 hours at 55±1° C. and agitator speed of 200 rpm, hydrolytic degradation was conducted. After cool-down of the solution to 20±1° C., 112 wt parts of a 10% aqueous sodium hydroxide were introduced at 4500 rpm of the agitator to attain pH=6.0 and agitator speed was raised to 8000 rpm until the gel-forming point was reached at pH=6.7.

Product was 4553 wt parts of a stable gel of the chitosan salt with jelly consistency containing 1.95 wt % of chitosan characterized by Mᵥ=320 kD, P_{d}=3.97, DD=78.0%.

### EXAMPLE 11

4439 wt parts of a 0.5% aqueous hydrochloric acid and 100 wt parts of the initial powdered chitosan with properties as in Example 4 were introduced to the reactor as in Example 5. Dissolution proceeded for 2 hours at 50 rpm. The solution was filtered using a press filter frame. 4530 wt parts of a solution containing 2 wt % of chitosan were obtained. The solution was introduced to a mixer equipped with a slow/fast agitator system and was subjected for 5 hours to a controlled hydrolytic degradation at 40±1° C. and 200 rpm of the agitator. After cooling to the 20±1° C., 94 wt parts of a 10% aqueous potassium hydroxide were introduced at 4500 rpm to attain pH=6.0. Next, agitation was continued at 8000 rpm till the gel-forming point at pH=6.54 was reached.

4618 wt parts of a stable chitosan gel were obtained, having a jelly consistency, containing 1.96 wt % of chitosan, characterized by Mᵥ=254 kD, P_{d}=3.54, DD=82.2%.

### EXAMPLE 12

4348 wt parts of 1.0% aqueous lactic acid and 100 wt parts of the initial powdered chitosan were introduced to the reactor as in Example 5. The chitosan was characterized by: Mᵥ=240 kD, P_{d}=3.49, DD=80.2%, content of insolubles 0.25% and moisture 11.0%. Dissolution proceeded for 2 hours at 50 rpm. The solution was filtered, using a frame filter press. 4440 wt parts of 2 wt % chitosan solution were obtained. The solution was introduced to a mixer equipped with a slow/fast agitator system and during 8 hours subjected to controlled hydrolytic degradation at 70±1° C. and 200 rpm of the agitator. The mixer content was next cooled over 15 minutes to 21° C. and then 114 wt parts of a 10% aqueous sodium hydroxide were introduced at 20±1° C. and 4500 rpm to attain pH=6.0. Afterwards, the agitator speed was increased to 8000 rpm and the mixture agitated until the gel-forming point was reached at pH=6.72.

4554 wt parts of a stable chitosan salt gel were obtained with a jelly consistency, containing 1.95 wt % of chitosan, characterized by: Mᵥ=140 kD, P_{d}=3.57, DD=80.2%.

### EXAMPLE 13

5030 wt parts of 0.5% aqueous lactic acid and 100 wt parts of the initial powdered chitosan were introduced to the reactor as in Example 5. The chitosan was characterized by Mᵥ=360 kD, P_{d}=3.36, DD=84.6%, content of insolubles 0%, moisture 10.4%. The dissolution proceeded for 2 hours at 50 rpm. The chitosan solution was filtered using a frame filter press. 5124 wt parts of a solution, containing 1.75 wt % of chitosan were obtained. The solution was introduced to a mixer equipped with a slow/fast agitator system and then a solution of hydrogen peroxide was added in the amount of 0.2 wt calculated on chitosan. The controlled oxidative degradation of the chitosan proceeded for 60 minutes at 20° C. and 120 rpm. Next, 309 wt parts of 10% aqueous sodium hydroxide were introduced at 20±1° C. and 4500 rpm to attain pH=6.0. The agitator speed was then raised to 8000 rpm and the agitation continued until the gel-forming point at pH=6.72 was reached.

5434 wt parts of the chitosan salt gel, having a jelly consistency were obtained, the gel containing 1.65 wt % of chitosan, characterized by: Mᵥ=150, P_{d}=3.50, DD=84.6%.

### EXAMPLE 14

4390 wt parts of a 2.0% aqueous acetic acid and 100 wt parts of the initial powdered chitosan with properties as in Example 13 were introduced to the reactor as in Example 1. The dissolution was conducted for 2 hours at 50 rpm of the agitator. The solution was filtered using a frame filter press. 4485 wt parts of the solution with 2.0 wt % content of chitosan were obtained. The solution was introduced to a mixer equipped with a slow/fast agitator system and a solution of hydrogen peroxide in the quantity of 0.2 wt % on chitosan was added. The controlled oxidative degradation proceeded for 150 minutes at 20° C. and 120 rpm. Next, 93 wt parts of 10% aqueous potassium hydroxide were introduced at 20±1° C. and 4000 rpm to attain pH=6.0. The mixture next agitated at 8000 rpm until the gel-forming point at pH=6.72 was reached.

5562 wt parts of a stable chitosan salt gel were obtained, the gel having a jelly consistency and containing 1.96 wt % of chitosan, characterized by Mᵥ=90 kD, P_{d}=3.65, DD=84.6%.

### EXAMPLE 15

4348 wt parts of 1% aqueous hydrochloric acid and 100 wt parts of the initial powdered chitosan with properties as in Example 12, were introduced to the reactor as in Example 5.

Dissolution proceeded for 2 hours at 50 rpm of the agitator and the solution was filtered using a frame filter press. 4440 wt parts of a solution having 2 wt content of chitosan was obtained. The solution was introduced to a mixer equipped with a slow/fast agitator system. Hydrogen peroxide was added in the amount of 0.2 wt % on chitosan. The controlled oxidative degradation was conducted for 125 minutes at 20° C. and 120 rpm of the agitator. Next, 91 wt parts of 10% aqueous potassium hydroxide were introduced at 20±1° C. and 4000 rpm to attain pH=6.0. The mixture was then agitated at 8000 rpm to reach the gel-forming point at pH=6.72.

4550 wt parts of a stable chitosan salt gel, having a jelly consistency were obtained, containing 1.95 wt % of chitosan, characterized by Mᵥ=40 kD, P_{d}=3.52, DD=80.2%.

### EXAMPLE 16

To glasteel reactor 350.6 kg sterile water and 5.5 kg chitosan added. At agitator speed of 52 rpm and room temperature, reactants were mixed for 8 minutes. Reactor jacket temperature was then set at 28° C. Three minutes later, agitator speed increased to 95 rpm and

1. A chitosan-calcium (I) complex, comprising: calcium (II) ions bound to a gel of a chitosan salt, wherein said complex contains ≧0.5 wt % chitosan having an average molecular weight≧10 kD, a polydispersity≧2.0, deacetylation degree≧65% and wherein said complex has a water retention value≧300%, pH≦6.9 and a calcium (II) ion content≧0.1 wt % relative to chitosan.

2. A chitosan-calcium complex according to claim 1, wherein said calcium (II) ions are bound with the chitosan gel by coordinate bonds or hydrogen bonds.

3. A chitosan-calcium (II) complex according to claim 1, wherein said complex is water soluble.

4. A method to produce a chitosan-calcium complex from a gel of a chitosan salt, comprising the steps of:
a) providing a suspension containing ≧0.01 wt % chitosan gel, said gel having an average polymerization degree≧10 kD, a polydispersity≧2.0, and deacetylation degree≧65%; and
b) mixing said chitosan gel with ≧0.01 wt % calcium (II) salt to form said complex;
wherein said complex has a water retention value≧300% and a pH≦6.9.

5. A method according to claim 4, wherein said calcium (II) salt is selected from the group consisting of calcium chloride and calcium acetate.

6. A method according to claim 5, wherein said calcium (B) salt concentration is 10-50 wt % relative to chitosan.

7. A method according to claim 4, wherein said mixing step is carried out at a temperature≧10° C.

8. A method according to claim 7, wherein said mixing step is carried out at a temperature between 20° C. and 40° C.

9. A chitosan-calcium (II) complex prepared according to the method of claim 4.

10. A method of preparing chitosan salt gels, comprising the steps of:
a) degrading chitosan in an aqueous acidic solution with enzymes, said solution having a chitosan concentration of ≧0.5 wt % for a desired time and at a desired temperature;
b) deactivating said enzymes after said desired time is completed;
c) adding an aqueous basic solution to said enzyme/aqueous chitosan mixture to attain 4.0≦pH≦6.0; and
d) continuously mixing said mixture until a gel of a chitosan salt forms.

11. A method according to claim 10, wherein said gel forms at 6.3≦pH≦6.9.

12. A method according to claim 10, wherein said aqueous acidic solution comprises an acid selected from the group consisting of hydrochloric acid, acetic acid and lactic acid.

13. A method according to claim 10, wherein said enzymes are selected from the group consisting of chitanases, cellulases and xylanases.

14. A method according to claim 10, wherein said aqueous basic solution comprises a member selected from the group consisting of sodium hydroxide, potassium hydroxide, sodium carbonate and potassium carbonate.

15. A method according to claim 10, wherein the concentration of chitosan in said aqueous acidic solution is between about 1 wt % and 3 wt %.

16. A method according to claim 10, wherein said degrading step is carried out at a temperature≧10° C.

17. A method according to claim 10, wherein said degrading step is carried out at a temperature between about 20° C. and 60° C.

18. A method according to claim 10, wherein said deactivating step is carried out at a temperature≧70° C.

19. A method according to claim 10, wherein said aqueous basic solution has a concentration of between about 5 wt % and 10 wt %.

20. A method according to claim 10, wherein said method is a batch process.

21. A method of preparing a gel of a chitosan salt, comprising the steps of:
a) degrading chitosan hydrolytically, said chitosan being dissolved in an aqueous acidic solution, said solution having a chitosan concentration of ≧0.5 wt % for a desired time and at a desired temperature;
b) adding an aqueous basic solution to the mixture of step a) to attain 4.0≦pH≦6.0; and
c) continuously mixing the product of step b) until a gel of a chitosan salt forms.

22. A method according to claim 21, wherein said step a) utilizes an acid selected from the group consisting of hydrochloric acid and chloroacetic acid.

23. A method according to claim 22, wherein the concentration of said acid used is at least 0.01 wt %.

24. A method according to claim 21, wherein step a) is carried out at a temperature of ≧20° C.

25. A method according to claim 24, wherein said temperature is between 40° C. and 80° C.

26. A method according to claim 21, wherein said aqueous acidic solution comprises hydrochloric acid, acetic acid or lactic acid.

27. A method according to claim 24, wherein said aqueous acidic solution has a chitosan concentration of between 1 wt % and 3 wt %.

28. A method according to claim 21, wherein said aqueous basic solution comprises a base selected from the group consisting of sodium hydroxide, potassium hydroxide, sodium carbonate and potassium carbonate.

29. A method according to claim 28, wherein said aqueous basic solution has a concentration of 5 wt % to 10 wt %.

30. A method according to claim 21, wherein said gel forms at 6.3≦pH≦6.9.

31. A method according to claim 21, wherein said method is a batch process.

32. A method according to claim 21, wherein said chitosan concentration in said aqueous acidic solution is between 1 wt % and 3 wt %.

33. A method of preparing a chitosan salt gel, comprising the steps of:
a) degrading chitosan with an oxidizing agent, said chitosan being dissolved in an aqueous acidic solution, said solution having a chitosan concentration of ≧0.5 wt % for a desired time and at a desired temperature;
b) adding an aqueous basic solution to the mixture of step a) to attain 4.0≦pH≦6.0; and
c) continuously mixing the product of step b) until a gel of a chitosan salt forms.

34. A method according to claim 33, wherein said oxidizing agent is selected from the group consisting of hydrogen peroxide and sodium perborate.

35. A method according to claim 33, wherein said aqueous acidic solution comprises a member of the group consisting of hydrochloric acid, acetic acid and lactic acid.

36. A method according to claim 33, wherein said concentration of chitosan is between 1 wt % and 3 wt %.

37. A method according to claim 33, wherein the concentration of said oxidizing agent is ≧0.001 wt %.

38. A method according to claim 37, wherein the concentration of said oxidizing agent is between 0.01 and 0.5 wt %.

39. A method according to claim 33, wherein said aqueous basic solution comprises a member selected from the group consisting of sodium hydroxide, potassium hydroxide, sodium carbonate and potassium carbonate.

40. A method according to claim 39, wherein said aqueous basic solution has a concentration of between 5 wt % and 10 wt %.

41. A method according to claim 33, wherein said gel forms at 6.3≦pH≦6.9.

42. A method according to claim 33, wherein said method is a batch process.
